(19) United States
(12) Patent Application Publication
(10) Pub. No.: US 2022/0260567 A1
(43) Pub. Date: Aug. 18, 2022

(54) BIOSENSOR FOR POINT-OF-CARE DIAGNOSTIC AND ON-SITE MEASUREMENTS

(71) Applicant: EIR DIAGNOSTICS APS, Egå (DK)

(72) Inventors: Noemi ROZLOSNIK, Egå (DK); Michael DØSSING, Egå (DK)

(73) Assignee: EIR DIAGNOSTICS APS, Egå (DK)

(21) Appl. No.: 17/623,158

(22) PCT Filed: Jun. 26, 2020
(86) PCT No.: PCT/EP2020/068014

§ 371 Date: Dec. 27, 2021

(30) Foreign Application Priority Data
Jul. 5, 2019 (DK) PA 2019 70441
Apr. 23, 2020 (DK) PA 2020 70251

(51) Int. Cl. G01N 33/569; G01N 27/02; G01N 33/543; C12Q 1/00; C12Q 1/26

(52) U.S. Cl. CPC G01N 33/56983; G01N 27/026; C12Q 1/26; C12Q 1/005; G01N 33/5438

(57) ABSTRACT

A biosensor for detection of a target substance in a sample with impedance spectroscopy or impedance measurement on a single frequency.

The invention relates to an improved biosensor for fast, easy and reliable point-of-care diagnostics and on-site measurements using impedance spectroscopy.

In a global community, the outbreak of infectious diseases contributes to the fear of severe pandemics, and is a source of worry for the population. In recent years, emerging viruses such as e.g. Dengue virus, West Nile virus, and influenza A virus, have been a focus of massive attention.

Conventional diagnostic methods for detection of acute viral disease count e.g. polymerase chain reactions (PCR) and enzyme linked immunosorbent assays (ELISA). These diagnostic methods give reliable results, but require rather complicated procedures. PCR and ELISA diagnostic methods are time consuming, expensive, labour intensive, and require trained personnel and specialised laboratories.

The demands in modern medical diagnosis thus put forward an enormous need for faster and cheaper detection methods for detecting viral infections, which give a prompt result. If it had been possible to efficiently diagnose humans for Swine-Origin influenza A (H1N1)-virus, back in 2009, treatment could have been applied quickly and the flu might not have been so widespread.

Further, the demands for a fast detection of bacteria or viruses and similar in food product or on surfaces, which is presumed bacteria and virus free also put forward a need for faster and cheaper detection methods, which give a prompt result.

Impedimetric biosensors are a class of biosensors used for detection of e.g. viruses using electrochemical impedance spectroscopy (EIS). When a target substance, such as e.g. a target molecule, binds to an impedance biosensor, it will induce a change in the electrode surface/liquid solution interface. The difference in the impedance measured before and after the target substance binds to the biosensor provides a direct indication of the amount of target substance in the sample. Thus, the presence of a target substance in a sample can be detected very efficiently with a biosensor using electrochemical impedance spectroscopy (EIS).

EIS measures the electrical impedance by imposing a small sinusoidal voltage at a certain frequency to an electrochemical cell and measuring the resulting current through the cell. The time dependent current-voltage ratio and the phase shift give the impedance. Since the impedance changes when the target analyte is captured on the surface of the electrodes, EIS represents a powerful method for probing the interfacial reactions of modified electrodes, providing a rapid approach for monitoring the dynamics of biomolecular interactions.

The impedance of a system is frequency dependent, and can be expressed as a complex number:

$$z ( f ) = {Z}_{0} ( cos ( ϕ ) + i sin ( ϕ ) ) \qquad [ 1 ]$$

where f is the frequency, Z₀ is the magnitude of the impedance, and ϕ is the phase shift. Thus, the impedance Z(f) consists of a real and an imaginary part. The graph, on which the imaginary part of the impedance is plotted against the real part (so-called “Nyquist Plot”), can be used to determine the most efficient frequency for the detection of the target binding.

Impedance biosensors are very promising for a variety of applications such as point-of-care diagnostics, on-site measurements, consumer test kits, bioprocess monitoring, water quality testing, and biowarfare agent detection, due to among others the advantage of label-free operation.

Especially point-of-care/on site applications, such as diagnostic or on-site testing, are promising in connection with impedance biosensors, as it brings the test conveniently and immediately to the patient, facilitating rapid treatment.

Existing technologies for point-of-care diagnostics, often based on laminar flow assays (LFA), are usually not very specific and have high detection limits.

Today, screening of samples for disease markers is expensive both in hours and reagents. Further, screenings are labour intensive and need to be conducted in specialised laboratories by trained personnel. Accordingly, it is an object of the invention to provide an improved, user friendly and cheap biosensor for point-of-care testing and onsite diagnostics.

### SUMMARY

Disclosed herein in a first aspect is a biosensor for detection of a target substance in a sample with impedance spectroscopy or impedance measurement on a single frequency. The biosensor may comprise a first non-conducting substrate comprising a primary substrate surface; and a conducting electrode layer comprising one or more conducting sub-layers including a first electrode sub-layer and a second electrode sub-layer. The conducting electrode layer may comprise a primary electrode surface and a secondary electrode surface, wherein the secondary electrode surface covers part of the primary substrate surface.

The biosensor may further comprise a probe layer bonded to part of the primary electrode surface, the probe layer being adapted for selectively binding of the target substance; and a second non-conducting substrate comprising a secondary substrate surface, wherein the secondary substrate surface of the second substrate and the primary substrate surface of the first substrate are interconnected such that the electrode layer and the probe layer are confined within an area defined by the first substrate and the second substrate. The electrode layer may comprise at least a first electrode pair with a primary electrode and a secondary electrode, wherein the probe layer is bonded to the primary electrode and/or the secondary electrode,

In the first aspect, the first electrode sub-layer is normally a conducting polymer electrode layer, and the second electrode sub-layer is positioned between the first electrode sub-layer and the probe layer, and is selected from the group of:
a redox material layer,
a polymerized redox material layer,
a functionalization layer,
a polymerized functionalization layer,
a graphene oxide layer,
a modified graphene oxide layer,
a nanoparticle layer.

Disclosed herein in a second aspect is a biosensor for detection of a target substance in a sample with impedance spectroscopy or impedance measurement on a single frequency or impedance measurement on a single frequency. The biosensor may comprise a first non-conducting substrate comprising a primary substrate surface; and a conducting electrode layer comprises one or more electrode sub-layers including a first electrode sub-layer. The electrode layer may comprise a primary electrode surface and a secondary electrode surface, wherein the secondary electrode surface covers part of the primary substrate surface.

The biosensor may further comprise a probe layer bonded to part of the primary electrode surface, the probe layer being adapted for selectively binding of the target substance; and a second non-conducting substrate comprising a secondary substrate surface, wherein the secondary substrate surface of the second substrate and the primary substrate surface of the first substrate are interconnected such that the electrode layer and the probe layer are confined within an area defined by the first substrate and the second substrate. The electrode layer may comprise at least a first electrode pair with a primary electrode and a secondary electrode, wherein the probe layer is bonded to the primary electrode and/or the secondary electrode,

In the second aspect, the first electrode sub-layer is selected from the group of:
a carbon electrode layer,
a glassy carbon electrode layer,
a graphene electrode layer,
a modified graphene oxide layer,
a two-dimensional transition-metal dichalcogenide layer,
a hexagonal boron nitride layer,
a graphene electrode layer comprising a redox material integrated therein,
a conducting polymer electrode layer comprising a redox material integrated therein,
a conductive polymer electrode layer comprising nanoparticles integrated therein,
a conductive polymer electrode layer comprising two-dimensional transition-metal dichalcogenides integrated therein,
a conductive polymer electrode layer comprising hexagonal boron nitride integrated therein.

The modified layer may have modifications by divalent ions, amine, thiol, and similar.

Examples of materials for the two-dimensional transition-metal dichalcogenides are Molybdenum Disulfide, Tungsten Disulfide, Rhenium Disulfide and similar materials.

Examples of materials for the first and the second substrate comprise glass, non-conducting polymers and similar. By non-conducting polymer is meant a polymer with a conductivity of less than 10⁻⁸ S/m and by conducting polymer is meant a polymer with a conductivity of at least 10 S/m.

Hereby is obtained a biosensor, which can be used for routine medical check-up, thereby greatly reducing the cost and time per analysis, allowing more people to be screened for different diseases both inside and outside of medical facilities. Thus, by the biosensor is provided an effective point-of-care biosensor to detect, control and confine the spread of virulent diseases in the near future.

Further, the biosensor provides a fast, sensitive, reliable and selective method for analysis of non-human sample types, such as e.g. water samples possibly containing bacteria or virus. This also included samples from food products.

The target substance, which is detectable with the biosensor according to the above-mentioned method, may be a target molecule, nanoparticles, viruses, bacteria, chemicals, antibiotics, fertiliser or medicines in general and is thus not limited to be a biological sample. A large variety of target substances may be selectively detected upon choosing the correct corresponding probes. The target substance may be an antibody and the probe layer an antigen probe layer comprising at least one antigen.

Electrochemical impedance spectroscopy also requires the binding event to be in close proximity to the surface. A direct link to the conductive electrode may therefore provide a defined distance of the binding event to the electric transducer.

The biosensor is an all-in-one device, where the sample is added to the biosensor instead of the biosensor having to be placed in a sample solution. Further as the biosensor normally includes both a primary electrode acting as the working electrode and a secondary electrode acting as a counter electrode, the need for additional electrodes—as used in conventional three electrode electrochemical cells—is absent. This reduces the amount of material needed for the device at the same time making the device highly adequate for mass production. Thus, production cost is reduced.

The electrodes in the biosensor are normally not metal based, which provides for a low material cost. The biosensor may consequently be used as a disposable device, which is advantageous when using the biosensor for point-of-care testing in a location, e.g. an airport, a school or a workplace, where multiple people need to be tested for a given virus and adequate cleaning of the biosensor between testing different people is impossible. When being a non-metal device, the used biosensor can be burned along with other types of biological waste, as there is no need for metal recovery.

Further, the biosensor only needs to be filled with the sample solution and connected electrically to an analysing unit. The detection results obtained by using the biosensor can thus be obtained in significantly shorter time with much less subsequent lab work as compared to standard testing methods such as e.g. ELISA and PCR. This makes the biosensor highly applicable in point-of-care methods and on-site measurements.

Thus, the biosensor according to the invention makes the process of detecting a specific target substance/object effortless due to the fact that the measurements now can take place on a localized electrode surface in contrast to targeting the point of interest in a solution e.g. using a standard three electrode electrochemical cell.

This is especially advantageous when detecting target substances in small amounts of samples.

In one or more examples, the primary electrode comprises a plurality of primary legs and the secondary electrode comprises a plurality of secondary legs, the primary legs and the secondary legs forms an interwoven pattern. The two electrodes are thereby in the same plane and in the same liquid making calibration of the two in relation to each other unnecessary.

In one or more examples, the second non-conducting substrate is provided with ports for inlet/outlet of the sample and/or for facilitating an electrical connection. The sample can thereby be added to the biosensor in a very easy manner, and likewise electrical connection to the electrodes in the biosensor is easily obtained by the ports.

In one or more examples, the first substrate and/or the second substrate is a non-conducting polymer substrate. Alternatively, the first substrate and/or the second substrate can be a glass substrate or similar. The first and the second substrate can be in the same material, which can be beneficial production wise, but it is not a requirement.

In one or more examples, the non-conducting polymer substrate is selected from the group of polystyrenes, polycarbonates, styrene acrylic copolymers, polyolefins, polyethylene terephthalates, polyethylene terephthalate glycol co-monomer, PC-blend (polycarbonate blend), ABS blend (Acrylonitrile Butadiene Styrene blend), PC-ABS blend, and cyclic olefin copolymers such as e.g. TOPAS 5013L (TOPAS Advanced Polymers, Germany).

In one or more examples, the one or more electrode sub-layers further comprises a second electrode sub-layer positioned between the first electrode sub-layer and the probe layer, wherein the second electrode sub-layer is selected from the group of:
a redox material electrode layer,
a polymerized redox material electrode layer,
a functionalization electrode layer,
a conducting polymer electrode layer,
a graphene oxide electrode layer,
a nanoparticle layer.

In one or more examples, the second electrode sub-layer is a redox/polymerized redox material electrode layer, wherein the redox or polymerized redox material electrode layer comprises a redox material/monomeric building block selected from the group of:
Methylene blue
Toluidine Blue O
Indigo carmine
Ferrocene
Vinyl-ferrocene
Hematein
Bipyridines
Oxidoreductases.

In one or more examples, Oxidoreductases is Laccase, Peroxidases, Hydroxylases, or Oxygenases Reductases.

In one or more examples, the second electrode sub-layer is a functionalization electrode layer comprising a conducting monomer or polymer with one or more functional groups. The one or more functional groups may be selected from amine, amide, hydroxyl, carboxylic acid, imine, thiol, azide, ether, alkene, alkyne, ester, phenyl, aldehyde, and/or alcohol groups.

In one or more examples, the nanoparticles may be selected from carbon nanotubes, carbon nanoribbons, metal nanoparticles, Titanium oxide, nano Silver, nano-scale iron or similar materials.

In one or more examples, the conducting polymer electrode layer comprises one or more conductive polymer micro-layers, wherein the polymer(s) are selected from the group of poly(3,4-ethylenedioxythiophene) (PEDOT), polypyrrole (PPy), poly(3,4-propylenedioxythiophene), triacetonamine (TAA), polyaniline (PANI), derivatives thereof and/or co-polymers thereof.

In one or more examples, the one or more conducting polymer(s) are selected from the group of PEDOT and PEDOT derivatives. The PEDOT is a polymerization of the building EDOT block. The PEDOT derivatives may contain one or more functional groups selected from the group of alcohols (OH), carboxylic acids (COOH), azides (N₃) and alkynes. These functional groups facilitate binding with the probe layer.

In one or more examples, the one or more conducting polymer electrode micro-layers comprises a first conductive polymer micro-layer and a second conducting polymer micro-layer, wherein the first conductive polymer micro-layer is PEDOT and the second conducting polymer micro-layer is a PEDOT-derivative.

The biosensor may comprise a linker bonded to the electrode layer, the linker being adapted for improving the selectively binding of the target substance to the probe layer. The linker may be bonded to the electrode layer by one or more of:
Ultraviolet light assisted binding,
Chemical binding,
Adsorption on the first electrode sub-layer,

The biosensor may comprise a probe bonded to the electrode layer. The probe may be bonded to the electrode layer by one or more of:
Ultraviolet light assisted binding,
Chemical binding,
Adsorption on the electrode sub-layer.
Hybridization with the linker.

In one or more examples, the chemical binding is one of:
Carbonyldiimidazole (CDI) chemistry,
Succinimidyl 4-(Nmaleimidomethyl) cyclohexane-1-carboxylate (SMCC) chemistry,
1-ethyl-3-(3-dimethylaminopropyl) Carbodiimide (EDC) chemistry,
N,N′-Dicyclohexylcarbodiimide (DCC) chemistry,
Thiol chemistry,
Silane chemistry
Click chemistry.

The EDC and DCC chemistry may be supplemented with an N-Hydroxysuccinimide (NHS) or Sulfo-NHS ester.

The linker may be bonded to the electrode layer by hybridization of the linker with a complemented DNA modified aptamer, e.g. using a spacer between the aptamer and the electrode sub-layer.

In one or more examples, the probe layer comprises one or more entities selected from the group of oligonucleotide aptamers, such as ssDNA aptamers, and RNA aptamers; modified oligonucleotides; peptide aptamers; nanobodies, antigens; or antibodies.

Probes of aptamers, modified oligonucleotides and/or peptides may be superior substitute to probes comprising antibodies in immunoassays, since aptamers, oligonucleotides and peptides have a higher stability, affinity, and specificity compared to antibodies in immunoassay. As aptamers, modified oligonucleotides and peptides are smaller in size than their antibody counter parts, the target substances captured by an aptamer, oligonucleotide and/or peptide probe will be much closer to the polymer layer than if using antibodies as probes. As a consequence, the change in the impedance signal due to the capture of the target substance by an aptamer, oligonucleotide and/or peptide probe will be much larger, enabling a more precise detection result, and higher sensitivity.

When the probe layer is an antigen probe layer, the antigen probe layer normally comprises at least one antigen, which is adapted for forming bonds with a corresponding antibody. By antigen is meant a molecule or molecular structure, such as may be present at the outside of a pathogen, that can be bound to by an antigen-specific antibody (Ab) or B cell antigen receptor (BCR). The presence of antigens in the body normally triggers an immune response. Antigens are “targeted” by antibodies produced by the triggering of the immune response. An antibody is specifically produced by the immune system in the human to match an antigen after cells in the immune system come into contact with it; this allows a precise identification or matching of the antigen and the initiation of a tailored response. Antigens may be proteins, peptides (amino acid chains) and polysaccharides (chains of monosaccharides/simple sugars). Lipids and nucleic acids may become antigens when combined with proteins and polysaccharides.

An antibody may be said to “match” the antigen in the sense that it can bind to it due to an adaptation in a region of the antibody. Therefore, different antibodies are produced, each being able to bind a different antigen while sharing the same basic structure. In most cases, an adapted antibody can only react to and bind one specific antigen; in some instances, however, antibodies may cross-react and bind more than one antigen.

By using an antigen probe layer, the biosensor may be employed to test bodily fluids for the presence of specific antibodies. If specific antibodies are detected, it may be concluded that a patient has (previously) been infected with e.g. a specific viral or intracellular bacterial infection. The presence of tumours inside the body may also result in the generation of antigens.

Also, if the antigen is a smaller part of the pathogen, the corresponding antibody enabling a small distance between the target antibody substance captured by an will be close to the polymer layer. As a consequence, the change in the impedance signal due to the capture of the target antibody substance by an antigen will be large, enabling a more precise detection result, and higher sensitivity.

In one or more examples, the electrode layer comprises a second electrode pair comprising a second primary electrode and a second secondary electrode. The probe layer is normally not bonded to the secondary electrode pair, thereby serving as a reference electrode. Alternatively, a non-target specific probe layer may be bonded to the secondary electrode pair, thereby serving as a reference electrode.

Disclosed herein is further in a third aspect, the use of a biosensor for point-of-care measurement and/or on-site detecting of a target substance in a liquid sample such as e.g. water, blood, urine, saliva, food product or a sample from a test surface. When testing e.g. human fluidic samples, the target substance may be an antibody and the probe layer an antigen probe layer comprising at least one antigen.

Disclosed herein is further in a fourth aspect, the use of a biosensor for point-of-care measurement and/or on-site detecting of a target substance in a liquid sample obtained during process and/or quality control measurements, during manufacturing of medicine, during manufacture of agents for therapy, or during a content control process in connection with food preparation.

Also disclosed herein is a system for detection of a target substance in a sample, the system comprising a biosensor, and an analysing unit for measuring changes in the impedance over the first and/or second electrode pair before and after applying the sample to the biosensor. By analysing unit is meant an apparatus for measuring the current over and/or imposing a current through the system, e.g. an apparatus which imposes a small sinusoidal voltage at a certain frequency to the biosensor and measures the resulting current through the biosensor. Hereby is obtained a fast, sensitive, reliable and selective impedance biosensor-based system for fast, cheap, and reliable point-of-care diagnostics and on-site measurements.

The system may further comprise connectors for operational connection between the analysing unit and the biosensor. Also, the system may comprise a touch display unit and a microcomputer for controlling the system and displaying the measured changes in the impedance. The display unit, the microcomputer and the analysing unit may be combined in one item and thereby making a standalone system.

### BRIEF DESCRIPTION OF THE DRAWINGS

FIGS. 1a-b are schematic illustrations of a biosensor with a non-limiting electrode design according to the invention displayed in a perspective view with (la) and without (1b) the second substrate layer.

FIG. 2 shows an enlargement of the electrode of the biosensor in FIGS. 1a-b displayed in a perspective view.

FIG. 3a-d shows examples of different enlargements of the electrode of the biosensor in FIGS. 1a-b displayed in a side view.

FIG. 4 shows the structure of four different monomer building blocks; A) EDOT, B) EDOT-OH, C) EDOT-COOH, and D) EDOT-N₃.

FIG. 5a shows an example of the biosensor viewed from the top and FIG. 5b shows the second substrate layer in a perspective view.

FIGS. 6a-b illustrate different working/counter electrode design options.

### DETAILED DESCRIPTION

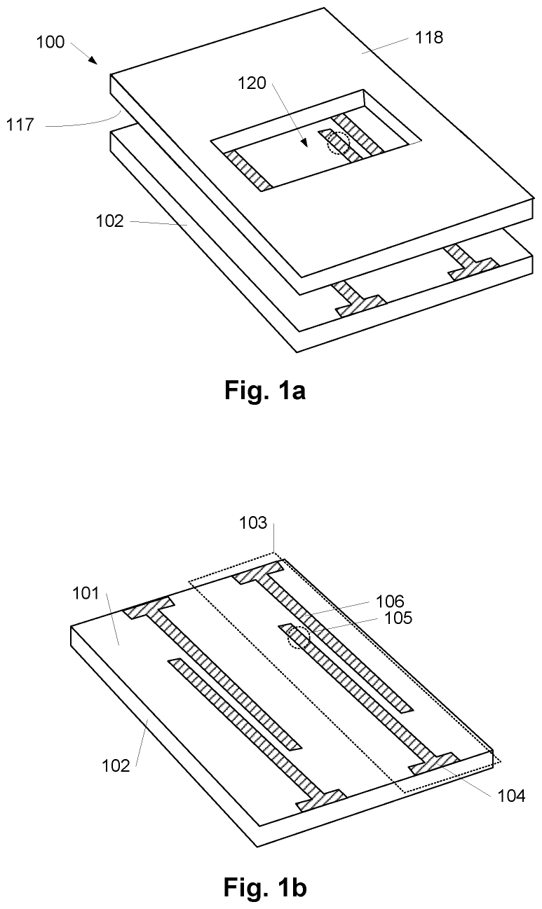

FIG. 1a-b illustrate the basic layout of a biosensor 100 according to the invention as seen in a perspective view with (FIG. 1a) and without (FIG. 1b) the second substrate layer.

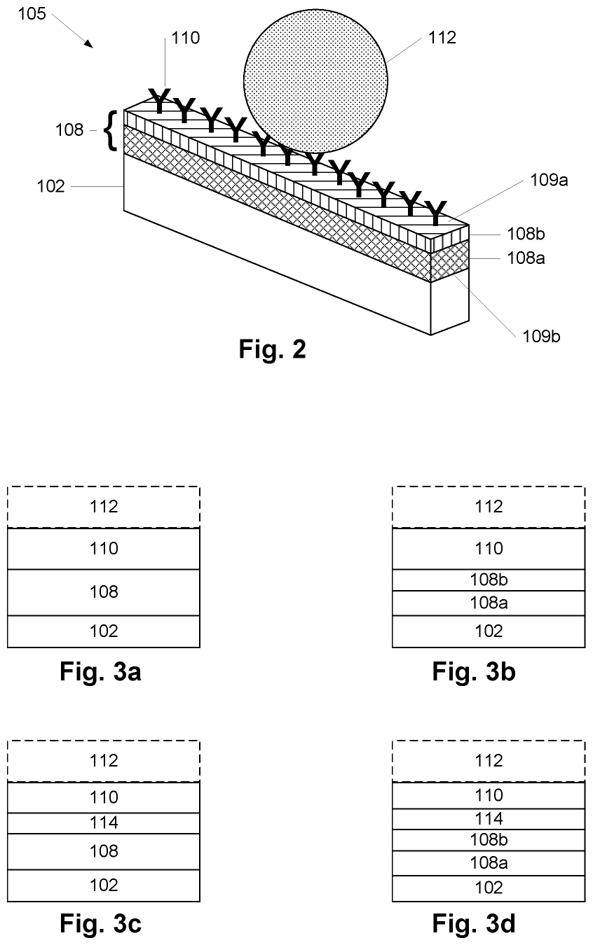

The biosensor 100 for detection of a target substance in a sample comprises a first non-conducting substrate 102 comprising a primary substrate surface 101. The biosensor 100 further comprises a conducting electrode layer 108 comprising one or more conducting sub-layers. The conducting electrode layer 108 comprises a primary electrode surface 109a and a secondary electrode surface 109b, wherein the secondary electrode surface 109b covers part of the primary substrate surface 101. The primary electrode surface 109a may also be seen as an upper surface of the electrode layer 108 and the secondary electrode surface 109b as a lower surface of the electrode layer 108. Some of the possible electrode sub-layers 108a, 108b are shown in FIG. 2. More sub-layers may be envisioned and a further division of each sub-layer into micro-layers together constituting one sub-layer could also be present in the biosensor.

The biosensor further comprises a probe layer 110 bonded to part of the primary electrode surface 109a. A second non-conducting substrate 118 comprising a secondary substrate surface 117 is also normally present in the biosensor. The secondary substrate surface 117 of the second substrate 118 and the primary substrate surface 101 of the first substrate 102 are interconnected such that the electrode layer 108 and the probe layer 110 are confined within an area defined by the first substrate 102 and the second substrate 118.

The electrode layer 108 comprises at least a first electrode pair 103, the first electrode pair comprising a primary electrode 104 and a secondary electrode 106 as shown in FIG. 1b. The probe layer 110 is normally bonded to the primary electrode 104 and/or the secondary electrode 106 of the at least first electrode pair 103.

On top of the first substrate layer 102 and the electrodes 104, 106 is normally a second substrate layer 118 as shown in FIG. 1a. The two substrate layers 102, 118 are of a non-conducting material such as a non-conducting polymer, glass or similar. Some examples of non-conducting polymers are polystyrenes, polycarbonates, styrene acrylic copolymers, polyolefins, polyethylene terephthalates, polyethylene terephthalate glycol co-monomer, PC-blend, ABS blend, PC-ABS blend, and cyclic olefin copolymers such as e.g. TOPAS 5013L (TOPAS Advanced Polymers, Germany).

The second substrate layer 118 may have an opening 120 forming a channel allowing samples to come in contact with the electrodes 104, 106.

The electrode layer 108 comprises a first electrode sub-layer 108a, which may be the only electrode sub-layer or one out of a number of electrode sub-layers. The first electrode sub-layer 108a may be made of a number of conducting materials including:
a carbon electrode layer,
a glassy carbon electrode layer,
a graphene electrode layer,
a modified graphene oxide layer,
a two-dimensional transition-metal dichalcogenide layer,
a hexagonal boron nitride layer,
a graphene electrode layer comprising a redox material integrated therein,
a conducting polymer electrode layer comprising a redox material integrated therein,
a conductive polymer electrode layer comprising nanoparticles integrated therein,
a conductive polymer electrode layer comprising two-dimensional transition-metal dichalcogenides integrated therein,
a conductive polymer electrode layer comprising hexagonal boron nitride integrated therein.

By modified graphene oxide layer is included modifications using divalent ions, amine, thiol, or similar.

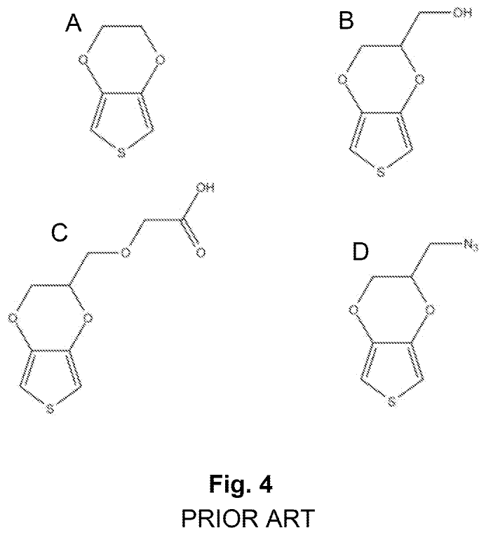

The electrode layer 108 may also comprise a second electrode sub-layer 108b positioned between the first electrode sub-layer 108a and the probe layer 110 as shown in FIG. 4. The second electrode sub-layer 108b may be selected from the group of:
a redox material electrode layer,
a polymerized redox material electrode layer,
a functionalization electrode layer,
a conducting polymer electrode layer,
a graphene oxide electrode layer.

The terms “sub-layer” is to be understood as a layer bonded together with another sublayer such that it is still identifiable as a layer. The term “integrated therein” is to be understood as a material integrated into one combined layer/sub-layer.

As an alternative to the above mentioned combinations of first and second sub-layers 108a, 108b, the biosensor may also comprise an electrode layer 108 comprising a first electrode sub-layer 108a being a conducting polymer electrode layer, and a second electrode sub-layer 108b selected from the group of:
a redox material layer,
a polymerized redox material electrode layer,
a functionalization layer,
a polymerized functionalization layer,
a graphene oxide layer
a modified graphene oxide layer
a nanoparticle layer.

The second sub-layer 108b will be positioned between the first electrode sub-layer 108a and the probe layer 110. If the second electrode sub-layer 108b is a redox/polymerized redox material electrode layer, the redox or polymerized redox material electrode layer may comprise a redox material/monomeric building block selected from the group of:
Methylene blue
Toluidine Blue O
Indigo carmine
Ferrocene
Vinyl-ferrocene
Hematein
Bipyridines
Oxidoreductases.

In one or more examples, Oxidoreductases is Laccase, Peroxidases, Hydroxylases, or Oxygenases Reductases.

If the second electrode sub-layer 108b is a functionalization electrode layer, it may comprise a conducting monomer or polymer with one or more functional groups. The one or more functional groups may be selected from amine, amide, hydroxyl, carboxylic acid, imine, thiol, azide, ether, alkene, alkyne, ester, phenyl, aldehyde, and/or alcohol groups.

Non-limiting examples of suitable conducting polymers for the first electrode sub-layer of the second electrode sub-layer include polypyrrole (PPy), poly(3,4-ethylenedioxythiophene) (PEDOT), poly(3,4-propylenedioxythiophene), triacetonamine (TAA), polyaniline (PANI), derivatives thereof and/or co-polymers formed by two or more of the monomeric units in the mentioned polymer examples.

Non-limiting examples of functional groups in the derivatives, e.g. the PEDOT derivatives, are alcohols (OH), carboxylic acids (COOH), azides (N₃) and alkynes. FIG. 4 shows 3,4-ethylenedioxythiophene (EDOT) (FIG. 4A) and the OH, COOH, and N₃ based EDOT-derivatives; EDOT-OH (FIG. 4B) EDOT-COOH (FIG. 4C) and EDOT-N₃ (FIG. 4D) used as the monomeric building block for PEDOT, PEDOT-OH, PEDOT-COOH, and PEDOT-N₃, respectively.

The conducting polymer electrode layer may comprise one or more conductive polymer micro-layers. Thus, the electrode layer 108 may comprise one or more conducting polymer electrode micro-layers comprising a first conductive polymer micro-layer and a second conducting polymer micro-layer, wherein the first conductive polymer micro-layer is PEDOT and the second conducting polymer micro-layer is a PEDOT-derivative.

Thus, one sub-layer of a conducting polymer or multiple micro-layers of the same or different conducting polymers may be present in the biosensor.

FIG. 2 shows an enlarged view 105 of part of an example of a primary electrode 104 functioning as the working electrode, wherein the polymer layer 108 coated onto the first substrate layer 102 comprises two sub-layers.

Bonded to the polymer layer 108 is a probe layer 110 comprising an entity, which binds selectively to a specific target substance 112. The target substance could be a virus, a protein, a cell, a peptide, a molecule (both organic and inorganic), a structured nanoparticle, an antibiotic, a fertiliser or similar. Thereby, when a sample, such as e.g. a blood sample, urine and saliva, water, or a sample obtained from a food product or a surface, possibly containing target substances 112 come in contact with the biosensor 100 (by adding the sample to the opening(s) 120 in the biosensor), the target substances 112 will form bonds, e.g. ionic bonds, hydrogen bonds or other electrostatic interaction bonds, with the probe 110. The sample may alternatively be obtained from during process and/or quality control measurements, during manufacturing of medicine, during manufacture of agents for therapy, or during a content control process in connection with food preparation.

The probe layer 110 may comprise probe layer comprises one or more entities selected from the group of:
Aptamers,
DNA aptamers,
RNA aptamers
Oligonucleotides,
Peptides,
Peptide aptamers,
Nanobodies,
Modified oligonucleotides,
Antibodies,
Antigens.

Aptamers are peptides or oligonucleotides (RNA or single stranded DNA) which typically fold into a three-dimensional structure, and whose conformation is changing upon ligand binding. Novel aptamers can be developed using a process called SELEX (Systematic Evolution of Ligands by Exponential enrichment). It enables the selection of high-affinity nucleic acid sequences from a random pool of candidates. The oligonucleotide aptamers can easily be modified with signal moieties and can be produced at low cost. Thus, the probe 110 may be a biological entity or a synthetically produced replica and/or modification of such.

By antigen is meant a molecule or molecular structure, such as may be present at the outside of a pathogen, that can be bound to by an antigen-specific antibody (Ab) or B cell antigen receptor (BCR). The presence of antigens in the body normally triggers an immune response. Antigens are “targeted” by antibodies produced by the triggering of the immune response. An antibody is specifically produced by the immune system in the human to match an antigen after cells in the immune system come into contact with it; this allows a precise identification or matching of the antigen and the initiation of a tailored response. Antigens may be proteins, peptides (amino acid chains) and polysaccharides (chains of monosaccharides/simple sugars). Lipids and nucleic acids may become antigens when combined with proteins and polysaccharides. By using an antigen probe layer, the biosensor may be employed to test bodily fluids for the presence of specific antibodies. If specific antibodies are detected, it may be concluded that a patient has (previously) been infected with e.g. a specific viral or intracellular bacterial infection. The presence of tumours inside the body may also result in the generation of antigens.

The polymer sub-layer, which binds to the probe 110 is normally chosen such that it facilitates an improved binding capacity between the polymer sub-layer and the probe 110. Depending on production cost and productions lines, a single electrode sub-layer may be preferable over the double layering design shown in FIG. 2.

The biosensor may also comprise a linker 114 connecting the probe layer 110 to the electrode layer 108. Different examples of this is shown in FIG. 3a-d. The linker 114 may be bonded to the electrode layer 108 by one or more of:
Ultraviolet light assisted binding,
Chemical binding,
Adsorption on the first electrode sub-layer, and
Hybridization.

The linker may be bonded to the electrode layer 108 by chemical binding, where the chemical binding is one of:
Carbonyldiimidazole (CDI) chemistry,
Succinimidyl 4-(Nmaleimidomethyl) cyclohexane-1-carboxylate (SMCC) chemistry,
1-ethyl-3-(3-dimethylaminopropyl) Carbodiimide (EDC) chemistry,
N,N′-Dicyclohexylcarbodiimide (DCC) chemistry,
Thiol chemistry,
Silane chemistry
Click chemistry.

The EDC and DCC chemistry may be supplemented with an N-Hydroxysuccinimide (NHS) or Sulfo-NHS ester.

The linker 114 may alternatively be bonded to the electrode layer 108 by hybridization of the linker with 114 a complemented DNA modified aptamer, e.g. using a spacer between the aptamer and the electrode layer.

If a linker 114 is not used, the probe 110 may be bonded to the polymer layer 108 normally by flushing a liquid containing the probe through the wafer thereby leaving the probes 110 on the polymer layer 108. Alternatively and additionally, the probe 110 may be bonded to the electrode layer 108 by ultraviolet light assisted binding, chemical binding, adsorption on the first electrode sub-layer, and Hybridization. The chemical binding may be selected from one of:
Carbonyldiimidazole (CDI) chemistry,
Succinimidyl 4-(Nmaleimidomethyl) cyclohexane-1-carboxylate (SMCC) chemistry,
1-ethyl-3-(3-dimethylaminopropyl) Carbodiimide (EDC) chemistry,
Thiol chemistry,
Silane chemistry
Click chemistry.

The probe 110 is normally selectively chosen for binding with the target substance, e.g. a virus, to be detected in a given sample.

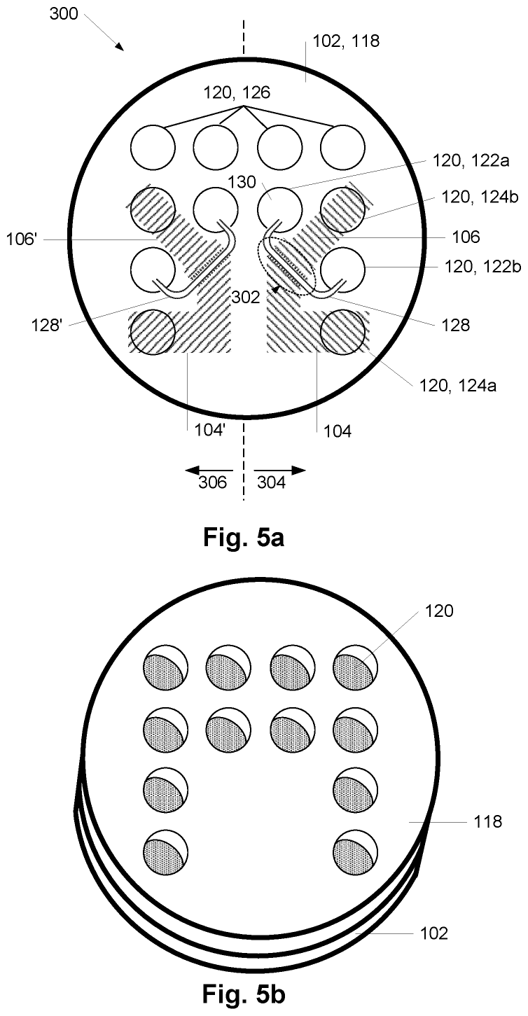

The second substrate layer 118 will normally contain access ports 120 for fluid inlets, outlets and/or electrical connections as shown in FIGS. 5a-b. An example of such is an access port in standard Luer lock size. The second substrate layer 118 is normally a non-conductive substrate layer (similarly to the first substrate layer 102) fabricated from e.g. polystyrenes, polycarbonates, styrene acrylic copolymers, polyolefins, polyethylene terephthalates, polyethylene terephthalate glycol co-monomer, PC-blend, ABS blend, PC-ABS blend, and cyclic olefin copolymers such as e.g. TOPAS 5013L (TOPAS Advanced Polymers, Germany).

The second substrate layer 118 and the first substrate layer 102 are preferably in the same material for reduced production costs. The second substrate layer 118 may also be patterned in a channel area situated opposite the patterned area 302 in the first substrate layer 102 when the two parts are assembled. This is beneficial production wise, as the second substrate layer 118 and the first substrate layer 102 can be produced in the same production line. Also, the patterned design forces the sample substances to distribute more evenly and thereby bind more efficiently to the probe 110 attached to the polymer 108.

The probe 110 can be applied before or after assembling the second substrate layer 118 and the first substrate layer 102. This is advantageous in mass production, because the target substance specificity of the biosensor can be selected after the production process. This can provide for an extremely fast production of biosensors with probe selectivity for a specific virus for example in case of an epidemic situation.

FIG. 5a shows an example of a biosensor 300 seen in a top down view where the second substrate is shown as a see-through object. FIG. 5b shows the second substrate 118 in a perspective view clearly showing the access ports 120 in the second substrate 118 for sample inlet/outlet and for providing electrical connections.

The biosensor 300 comprises a first substrate layer 102 and a second substrate layer 118, the latter comprising access ports 120 in standard Luer lock size. Two of the ports 122a, 122b provide inlet/outlet openings for the sample possibly containing target substances. Connection between the two inlet/outlet ports 122a, 122b is facilitated by a channel 128 formed in the second substrate layer 118 and/or the first substrate layer 102.

Electrical connection between the primary electrode 104 (acting as the working electrode) and the secondary electrode 106 (acting as the counter electrode) is provided through the electrode ports 124a and 124b, respectively, using connectors. The connectors further provide for operational connection between an analysing unit and the biosensor. By analysing unit is meant an apparatus for measuring the current over and/or imposing a current through the system, e.g. an apparatus which imposes a small sinusoidal voltage at a certain frequency to the biosensor and measures the resulting current through the biosensor.

A patterned electrode design is present in the sensing area 302 of the biosensor 300 where the primary electrode 104, the secondary electrode 106 and the sample channel 128 overlap, thereby creating an interwoven electrode pattern.

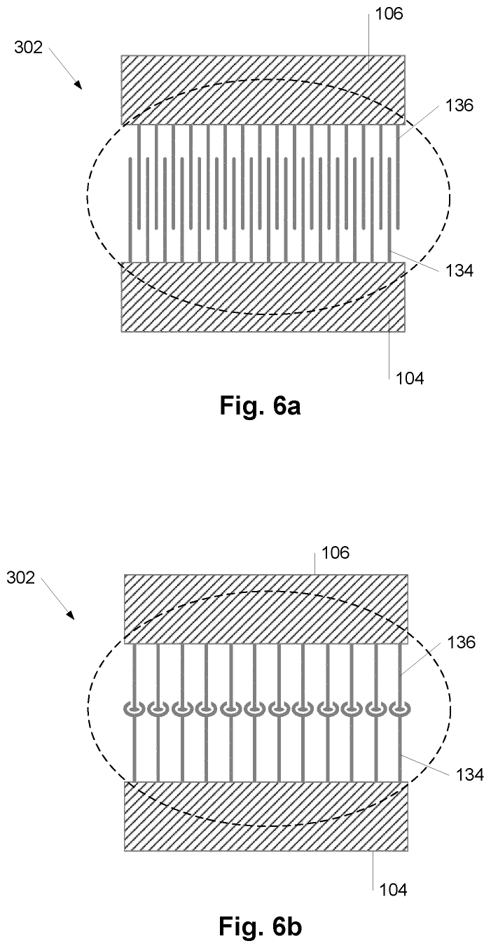

FIGS. 6a and 6b are enlargement top-down views of the interwoven electrode array design showing different examples of possible primary electrode 104 and secondary electrode 106 designs. In FIG. 6a, the primary electrode legs 134 and the secondary electrode legs 136 form a woven leg pattern. In FIG. 6b, a different design is shown and many other design options could also be possible.

The ports 126 seen as an upper line in FIG. 5a are in the shown example not used for detection of target substances. Instead the ports 126 represent the option that multiple electrode pairs 103 and sample channels 128 can be present. This will allow for simultaneous detection of more than one target substance by using different probes 110 attached to the polymer layer 108 in different electrode pairs 103.

Detecting several targets with one device is favourable for reducing cost, time and number of samples necessary for the test. In order to ensure that only one type of probe is attached to one set of electrode pairs 103, different polymers selectively forming bonds with specific probes may be used for the different electrode pairs 103. Alternatively, physically blocking access to all but one set of electrode pairs 103 could also ensure that the probe only binds to the electrode layer 108 in this set of electrode pairs 103.

Physically blocking access to all but one set of electrode pairs also allows for the use of the same electrode material in all the electrodes pairs, thereby reducing production costs and complications regarding different conductivities. The multiple port design shown in FIG. 5a makes it possible to bind different probes to different electrode pairs by using different ports connected two and two each by an individual sample channel 128.

The biosensor 300 shown in FIG. 5a has two symmetric sides; the measurement side 304 and the reference measurement side 306. The only difference between the electrodes 104, 106 and the sample channel 128 on the measurement side 304 and the electrodes 104′, 106′ and channel 128′ on the reference side 306 is that the measurement side electrodes 104, 106 has a probe 110 bonded to the electrode layer 108, whereas the reference side electrodes 104′, 106′ lack the probe.

When a sample containing a target substance is added to the channels 128, 128′ on both the measurement side 304 and the reference side 306, the target substances 112 will bind to the probe 110 on the electrodes 104, 106 on the measurement side 304, but not to the electrodes 104′, 106′ on the reference side 306. This will introduce a change in the impedance on the measurement side 304 but not on the reference side 306. The difference in the impedance measured on the measurement side 304 and the reference side 306 thereby provides a direct indication of the amount of target substance in the sample. Thus, the presence of a target substance 112 in a sample can be detected very efficiently with a biosensor according to the invention by using EIS.

Contributions to the impedance from target substances 112 binding directly to the electrode layer 108 are also eliminated by measuring the impedance both on the reference side 306 and the measurement side 304 of the biosensor 300.

The impedance biosensor incorporates both the primary electrode 104 and the secondary electrode 106 and does not necessitate an extra reference electrode. This makes the process of detecting a specific substance extremely simple due to the fact that the measurements can take place using only one electrode pair instead of the standard three-electrode electrochemical cell. This is especially advantageous when detecting target substances in small amounts of samples, as using a standard three-electrode electrochemical cell requires a relatively large sample volume in order to a reliable result.

The biosensor is easily mass-produced and may hold several other advantages such as high integration, low sample- and reagent volume, short analysis time, low sample waste and low material cost. Low material cost allows the biosensor to be used as a disposable device. This is advantageous, if the biosensor is used for point-of-care testing in a location, e.g. an airport, a school or a workplace, where multiple people need to be tested for a given virus and adequate cleaning of the biosensor in between testing different people is impossible.

Alternatively, the probe can be heated or treated with a high concentrated salt solution in order to release the target substance, whereby the biosensor can be used multiple times.

The biosensor shown in FIG. 5a is typically 3-10 cm in diameter and 0.5-2 cm thick. The sample volume required for obtaining a reliable result is approximately 10-200 μl.

The detection of target substances in a sample using the biosensor and EIS is an advantageous method as it eliminates the need for labelling the target substance due to the fact that the binding event is detected directly by a change in the surface properties of the electrode. Thus, impedance biosensors are favourable due to their high sensitivity and ability to perform label free detection. Labelling a bio-substance can drastically change its binding properties, thereby giving a highly variable detection results.

### REFERENCES

100 biosensor
101 primary surface of the first substrate layer
102 first substrate layer
103 electrode pair
104, 104′ primary electrode, acting as working electrode
105 part of the primary electrode
106, 106′ secondary electrode, acting as counter electrode
108 electrode layer
108a first electrode sub-layer
108b second electrode sub-layer
109a primary electrode surface
109b secondary electrode surface
110 probe
112 target substance
114 linker
117 secondary surface of the second substrate layer
118 second substrate layer
120 opening, port
122a, 122b port for the sample
124a, 124b port for providing electrical connection
126 port not in use
128, 128′ channel connecting sample ports
300 biosensor
302 sensing area of the biosensor
304 measurement side of the biosensor 300
306 reference side of the biosensor 300

1. A biosensor for detection of a target substance in a sample with impedance spectroscopy, the biosensor comprising:
a first non-conducting substrate comprising a primary substrate surface;
a conducting electrode layer comprising:
a first electrode sub-layer;
a second electrode sub-layer;
a primary electrode surface; and
a secondary electrode surface, wherein the secondary electrode surface covers part of the primary substrate surface;
a probe layer bonded to part of the primary electrode surface, the probe layer being adapted for selectively binding of a target substance; and
a second non-conducting substrate comprising a secondary substrate surface, wherein the secondary substrate surface and the primary substrate surface are interconnected such that the conducting electrode layer and the probe layer are confined within an area defined by the first non-conducting substrate and the second non-conducting substrate;
wherein the conducting electrode layer comprises a primary electrode and a secondary electrode, wherein the probe layer is bonded to the primary electrode and/or the secondary electrode,
wherein the first electrode sub-layer is a conducting polymer electrode layer, and
wherein the second electrode sub-layer is positioned between the first electrode sub-layer and the probe layer, and is selected from the group consisting of:
a redox material layer,
a polymerized redox material electrode layer,
a functionalization layer,
a polymerized functionalization layer,
a graphene oxide layer
a modified graphene oxide layer, and
a nanoparticle layer.

2. A biosensor for detection of a target substance in a sample with impedance spectroscopy, the biosensor comprising:
a first non-conducting substrate comprising a primary substrate surface;
a conducting electrode layer comprising:
an electrode sub-layer;
a primary electrode surface; and
a secondary electrode surface, wherein the secondary electrode surface covers part of the primary substrate surface;
a probe layer bonded to part of the primary electrode surface, the probe layer being adapted for selectively binding of a target substance; and
a second non-conducting substrate comprising a secondary substrate surface, wherein the secondary substrate surface and the primary substrate surface are interconnected such that the conducting electrode layer and the probe layer are confined within an area defined by the first non-conducting substrate and the second non-conducting substrate;
wherein the electrode layer comprises a primary electrode and a secondary electrode, wherein the probe layer is bonded to the primary electrode and/or the secondary electrode,
wherein the electrode sub-layer is selected from the group consisting of:
a carbon electrode layer,
a glassy carbon electrode layer,
a graphene electrode layer,
a modified graphene oxide layer,
a two-dimensional transition-metal dichalcogenide layer,
a hexagonal boron nitride layer,
a graphene electrode layer comprising a redox material integrated therein,
a conducting polymer electrode layer comprising a redox material integrated therein,
a conductive polymer electrode layer comprising nanoparticles integrated therein,
a conductive polymer electrode layer comprising two-dimensional transition-metal dichalcogenides integrated therein, and
a conductive polymer electrode layer comprising hexagonal boron nitride integrated therein.

3. The biosensor according to claim 2, wherein the conducting electrode layer further comprises a second electrode sub-layer positioned between the electrode sub-layer and the probe layer, wherein the second electrode sub-layer is selected from the group consisting of:
a redox material electrode layer,
a polymerized redox material electrode layer,
a functionalization layer,
a polymerized functionalization layer,
a conducting polymer electrode layer,
a graphene oxide electrode layer, and
a nanoparticle layer.

4. The biosensor according to claim 1, wherein the second electrode sub-layer is a redox/polymerized redox material electrode layer, wherein the redox/polymerized redox material electrode layer comprises a redox material/monomeric building block selected from the group consisting of:
Methylene blue,
Toluidine Blue O,
Indigo carmine,
Ferrocene,
Vinyl-ferrocene,
Hematein,
Bipyridines, and
Oxidoreductases.

5. The biosensor according to claim 4, wherein Oxidoreductases is Laccase, Peroxidases, Hydroxylases, or Oxygenases Reductases.

6. The biosensor according to claim 1, wherein the second electrode sub-layer is a functionalization layer comprising a monomer or polymer with one or more functional groups.

7. The biosensor according to claim 6, wherein the one or more functional groups are selected from amine, amide, hydroxyl, carboxylic acid, imine, thiol, azide, ether, alkene, alkyne, ester, phenyl, aldehyde, and/or alcohol groups.

8. The biosensor according to claim 1, wherein the target substance is an antibody and wherein the probe layer is an antigen probe layer comprising at least one antigen.

9. The biosensor according to claim 1, wherein the conducting polymer electrode layer comprises one or more conductive polymer micro-layers, wherein the polymer(s) are selected from poly(3,4-ethylenedioxythiophene) (PEDOT), polypyrrole (PPy), poly(3,4-propylenedioxythiophene), triacetonamine (TAA), polyaniline (PANT), derivatives thereof and/or co-polymers thereof.

10. (canceled)

11. The biosensor according to claim 1, wherein the primary substrate surface and/or the secondary substrate surface is a non-conducting polymer substrate, wherein the non-conducting polymer substrate is selected from the group consisting of polystyrenes, polycarbonates, styrene acrylic copolymers, polyolefins, polyethylene terephthalates, polyethylene terephthalate glycol co-monomer, PC-blend, ABS blend, PC-ABS blend, and cyclic olefin copolymers.

12. The biosensor according to claim 1, wherein the probe layer is bonded to the conducting electrode layer by one or more of:
Ultraviolet light assisted binding,
Chemical binding,
Adsorption on the electrode sub-layer,
Hybridization with a linker.

13. (canceled)

14. The biosensor according to claim 1, further comprising a linker connecting the probe layer to the conducting electrode layer, wherein the linker is bonded to the conducting electrode layer by one or more of:
Ultraviolet light assisted binding,
Chemical binding,
Adsorption on the first electrode sub-layer.

15. The biosensor according to claim 14, wherein when the linker is bonded to the conducting electrode layer is by chemical binding, the chemical binding is one of the group consisting of:
Carbonyldiimidazole (CDI) chemistry,
Succinimidyl 4-(Nmaleimidomethyl) cyclohexane-1-carboxylate (SMCC) chemistry,
1-ethyl-3-(3-dimethylaminopropyl) Carbodiimide (EDC) chemistry,
N,N′-Dicyclohexylcarbodiimide (DCC) chemistry,
Thiol chemistry,
Silane chemistry, and
Click chemistry.

16. The biosensor according to claim 15, wherein the EDC and DCC chemistry is supplemented with an N-Hydroxysuccinimide (NHS) or Sulfo-NHS ester.

17. The biosensor according to claim 1, wherein the probe layer comprises one or more entities selected from the group consisting of:
Oligonucleotide aptamers such as e.g. ssDNA aptamers, and RNA aptamers,
Modified oligonucleotides,
Peptide aptamers,
Nanobodies,
Antigen, and
Antibodies.

18. (canceled)

19. The biosensor according to claim 1, wherein the conducting electrode layer comprises a second primary electrode surface and a second secondary electrode surface, wherein a non-target specific probe layer is bonded to the secondary primary electrode surface and the second secondary electrode surface, thereby serving as a reference electrode.

20. A method of using a biosensor comprising:
directing a biosensor towards a sample, wherein the biosensor comprises:
a first non-conducting substrate comprising a primary substrate surface;
a conducting electrode layer comprising:
a first electrode sub-layer;
a second electrode sub-layer;
a primary electrode surface; and
a secondary electrode surface, wherein the secondary electrode surface covers part of the primary substrate surface;
a probe layer bonded to part of the primary electrode surface, the probe layer being adapted for selectively binding of a target substance in the sample; and
a second non-conducting substrate comprising a secondary substrate surface, wherein the secondary substrate surface and the primary substrate surface are interconnected such that the conducting electrode layer and the probe layer are confined within an area defined by the first non-conducting substrate and the second non-conducting substrate;
wherein the conducting electrode layer comprises a primary electrode and a secondary electrode, wherein the probe layer is bonded to the primary electrode and/or the secondary electrode,
wherein the first electrode sub-layer is a conducting polymer electrode layer, and
wherein the second electrode sub-layer is positioned between the first electrode sub-layer and the probe layer, and is selected from the group consisting of:
a redox material layer,
a polymerized redox material electrode layer,
a functionalization layer,
a polymerized functionalization layer,
a graphene oxide layer
a modified graphene oxide layer, and
a nanoparticle layer;
wherein the method further comprises:
performing either point-of-care measurement and/or on-site detecting of the target substance in the sample, wherein the sample is a liquid sample or a sample from a test surface.

21. A method of using a biosensor comprising:
directing a biosensor towards a sample, wherein the biosensor comprises:
a first non-conducting substrate comprising a primary substrate surface;
a conducting electrode layer comprising:
a first electrode sub-layer;
a second electrode sub-layer;
a primary electrode surface; and
a secondary electrode surface, wherein the secondary electrode surface covers part of the primary substrate surface;
a probe layer bonded to part of the primary electrode surface, the probe layer being adapted for selectively binding of a target substance in the sample; and
a second non-conducting substrate comprising a secondary substrate surface, wherein the secondary substrate surface and the primary substrate surface are interconnected such that the conducting electrode layer and the probe layer are confined within an area defined by the first non-conducting substrate and the second non-conducting substrate;
wherein the conducting electrode layer comprises a primary electrode and a secondary electrode, wherein the probe layer is bonded to the primary electrode and/or the secondary electrode,
wherein the first electrode sub-layer is a conducting polymer electrode layer, and
wherein the second electrode sub-layer is positioned between the first electrode sub-layer and the probe layer, and is selected from the group consisting of:
a redox material layer,
a polymerized redox material electrode layer,
a functionalization layer,
a polymerized functionalization layer,
a graphene oxide layer
a modified graphene oxide layer, and
a nanoparticle layer;
wherein the method further comprises:
performing either point-of-care measurement and/or on-site detecting of the target substance in the sample, wherein the sample is a liquid sample obtained during process and/or quality control measurements, during manufacturing of medicine, during manufacture of agents for therapy, or during a content control process in connection with food preparation.

22. A system for detection of a target substance in a sample, the system comprising:
a biosensor comprising:
a first non-conducting substrate comprising a primary substrate surface;
a conducting electrode layer comprising:
a first electrode sub-layer;
a second electrode sub-layer;
a primary electrode surface; and
a secondary electrode surface, wherein the secondary electrode surface covers part of the primary substrate surface;
a probe layer bonded to part of the primary electrode surface, the probe layer being adapted for selectively binding of a target substance in the sample; and
a second non-conducting substrate comprising a secondary substrate surface, wherein the secondary substrate surface and the primary substrate surface are interconnected such that the conducting electrode layer and the probe layer are confined within an area defined by the first non-conducting substrate and the second non-conducting substrate;
wherein the conducting electrode layer comprises a primary electrode and a secondary electrode, wherein the probe layer is bonded to the primary electrode and/or the secondary electrode,
wherein the first electrode sub-layer is a conducting polymer electrode layer, and
wherein the second electrode sub-layer is positioned between the first electrode sub-layer and the probe layer, and is selected from the group consisting of:
a redox material layer,
a polymerized redox material electrode layer,
a functionalization layer,
a polymerized functionalization layer,
a graphene oxide layer
a modified graphene oxide layer, and
a nanoparticle layer; and
an analyzing unit adapted for measuring changes in impedance over the primary electrode and the secondary electrode before and after applying the sample to the biosensor.

23. (canceled)

24. The system according to claim 22 further comprising:
connectors for operational connection between the analyzing unit and the biosensor; and
a touch display unit and a microcomputer for controlling the system and displaying the measured changes in the impedance.

25. (canceled)
